(19) United States
(12) Patent Application Publication
(10) Pub. No.: US 2010/0087845 A1
(43) Pub. Date: Apr. 8, 2010

(54) METHODS FOR AMELIORATING TISSUE TRAUMA FROM SURGICAL INCISIONS

(76) Inventors: Clifford Spiro, Naperville, IL (US); James Hicks, Naperville, IL (US)

Correspondence Address: STEVEN WESEMAN;ASSOCIATE GENERAL COUNSEL, I.P., CABOT MICROELECTRONICS CORPORATION, 870 NORTH COMMONS DRIVE, AURORA, IL, 60504, US

(21) Appl. No.: 12/449,085

(22) PCT Filed: Jan. 22, 2008
(86) PCT No.: PCT/US2008/000865

§ 371 Date: Jul. 22, 2009

Related U.S. Application Data
(60) Provisional application No. 60/881,893, filed on Jan. 22, 2007.

(51) Int. Cl. A61B 17/3211

(52) U.S. Cl. 606/167

(57) ABSTRACT

Methods for ameliorating tissue trauma from a surgical incision comprise making the surgical incision with a cutting instrument comprising a cutting instrument body defining two opposed sides and a direction of elongation, and having at least one cutting edge extending along the direction of elongation. The cutting edge defines an ultimate edge and two beveled faces adjacent the ultimate edge. The cutting edge of the cutting instrument has at least one characteristic selected from the group consisting of (a) a uniform ultimate edge having a maximum height deviation of 4 m or less along any 680 m segment of thereof; (b) each beveled face having a maximum height deviation of 4 m or less along any 680 m segment of thereof; and (c) each beveled face adjacent the ultimate edge having a root mean square surface roughness (Rq) of not more than about 200 nm. Improved cutting instruments are also provided.

### FIELD OF THE INVENTION

This invention pertains to methods for ameliorating tissue trauma from surgical incisions and for promoting healing of surgical incisions.

### BACKGROUND OF THE INVENTION

Surgical incisions necessarily cause some trauma or damage to the tissues through which the incision is made. Such trauma includes, for example, rupture of cells along the incision, abrasion of tissue due to drag from the cutting instrument used to make the incision, and tearing of tissue due to imperfections and unevenness of the cutting edge of the cutting instrument. The result of tissue trauma caused by surgical incisions can include increased rates of infection, increased scarring, increased time for wound closure, and the like.

Generally, cutting edges on cutting instruments, such as surgical blades, are manufactured by processing an appropriate feedstock to provide a cutting edge that has a beveled contour or profile in which the thickness diminishes toward the ultimate working edge. Conventional cutting edge-forming processes typically involve grinding operations that remove material in a gradient beginning at a distance from the ultimate edge to the ultimate edge itself, creating the beveled contour. The process of grinding generally involves contacting the feedstock with hard abrasive particles imbedded in a grinding wheel rotating about an axis, thereby mechanically abrading material from the feedstock. This grinding operation often is carried out with large abrasive particles that tend to leave relatively large gouges in the surface of the cutting edge or burrs along the cutting edge. Subsequent processes of honing and stropping are then used to remove burrs and reduce the depth of gouges on the cutting edge surface. Honing and stropping are both mechanical processes that remove the softer material of the cutting edge by abrasion.

A cutting edge may be characterized in several ways. For example, the thickness of the ultimate edge and/or the smoothness of the sides of the cutting edge determine, at least in part, the “sharpness” of the cutting edge. A thinner ultimate edge will generally encounter less resistance in parting of the material being cut, while smooth sides reduce drag and friction between the sides and the tissue in contact therewith during the incision. Sharpness is associated with a number of parameters, including the width if the ultimate edge of the blade and the uniformity of the ultimate edge, which can be assessed, for example, microscopically of using high band-pass edge roughness measurements of the blade edges (e.g., the high band-pass root mean square edge roughness). Another parameter impacting the performance and perceived sharpness of a cutting instrument is the smoothness and the contour of the sides of the cutting edge (e.g., of the beveled faces adjacent the ultimate edge of the cutting instrument). An irregular contour will lead to small (e.g., microscopic) points or protrusions that penetrate the material being cut before other parts of the cutting edge encounter the substrate. This leads to some degree of undesirable tearing rather than desirable slicing of the tissue. A cutting edge having a rough surface on the opposed sides of the instrument body, e.g., on the beveled faces adjacent the ultimate cutting edge, can abrade or tear material that passes over the sides of the cutting edge, as the material must be pushed aside to allow for passage of the cutting edge through the material being cut. The relative smoothness of the faces of a cutting instrument blade can be quantified, for example, by the deviation in height over a defined length of the ultimate edge and/or over a defined length of one or both faces of the cutting edge, as well as by the surface roughness of the edge and/or faces, for example, the average surface roughness (Ra) and/or root mean square surface roughness (Rq), which are well known in the art.

In the surgical arts, where the material being cut is living tissue, a sharp and smooth cutting edge in, for example, surgical scalpels, is of paramount interest. The trauma to living tissue caused by surgical incisions results from the work that must be imparted to the tissue in making the incision. The work required to pass a scalpel through tissue results from many factors, including edge sharpness, force applied to the blade, drag force (e.g., due to friction) acting on the sides of the blade, and the like. The tissue trauma caused by an incision can result in increased time required for healing, increased chance for infection, a limitation to the size of physiological structures that can be incised accurately, unsightly scarring, weakened tissue at the healed incision site, and the like.

In this regard, many efforts have been made to improve the performance of cutting instruments, particularly of surgical instruments such as scalpels. For example, cutting instruments have been fabricated from diamonds, rubies, and sapphires, which are very hard materials and can be manufactured with edges that are very thin. However, these materials are very expensive and difficult to fabricate. Their hardness is actually a disadvantage in medical operations, as they tend to fracture upon encountering hard structures such as bone, thus potentially leaving fragments in the operative subject. Metals are economically processed into surgical scalpels and the like. Difficulties with achieving sharp and smooth cutting edges have led to expedients, such as coating of cutting edges with friction-reducing materials, to reduce trauma resulting from the incisions. Such expedients can add costs and complicate the manufacturing process.

Due to the inherent tissue trauma caused by surgical incisions, there is an ongoing need for methods to ameliorate tissue trauma from surgical incisions and to promote healing of surgical incisions. The present invention provides such methods. These and other advantages of the invention, as well as additional inventive features, will be apparent from the description of the invention provided herein.

### BRIEF SUMMARY OF THE INVENTION

In one aspect, the present invention is directed to a method for ameliorating tissue trauma from a surgical incision. The method comprises making the surgical incision with a cutting instrument comprising a cutting instrument body having two opposed sides and a direction of elongation, and including at least one cutting edge extending along the direction of elongation. The cutting edge defines an ultimate edge and two beveled faces adjacent the ultimate edge. The cutting edge of the cutting instrument has at least one of the following characteristics: (a) the ultimate cutting edge has a maximum height deviation of about 4 μm or less along any 680 μm long segment thereof; (b) each beveled face adjacent the ultimate edge has a maximum height deviation of about 4 μm or less along any 680 μm long linear segment thereof; and (c) each beveled face adjacent the ultimate edge has a root mean square (RMS) surface roughness (Rq) of about 200 nm or less, as determined by surface metrology techniques that are well known in the art. Preferably, the ultimate cutting edge and/or the beveled faces adjacent thereto have a maximum height deviation of about 1 μm or less along any 680 μm linear segment thereof.

In another aspect, the present invention is directed to a method for promoting the healing of surgically incised tissue. The method comprises making a surgical incision with a cutting instrument preferably comprising a cutting instrument body having two opposed sides and a direction of elongation, and including at least one cutting edge extending parallel to the direction of elongation. The cutting edge defines an ultimate edge and two beveled faces adjacent the ultimate edge, wherein the cutting edge of the cutting instrument has at least one of the following characteristics: (a) the ultimate cutting edge has a maximum height deviation of about 4 μm or less along any 680 μm long segment thereof; (b) each beveled face adjacent the ultimate edge has a maximum height deviation of about 4 μm or less along any 680 μm long linear segment thereof; and (c) each beveled face adjacent the ultimate edge has a RMS surface roughness of about 200 nm or less.

Other aspects of the invention include a method of ameliorating scarring of surgically incised tissue, a method of ameliorating inflammation during healing of surgically incised tissue, a method ameliorating swelling during healing of surgically incised tissue, a method of promoting closure of a surgical incision, a method for promoting reepithelialization of surgically incised tissue, a method of promoting tissue strength in a healing and/or healed surgically incised tissue, and a method of ameliorating tissue morbidity in surgically incised tissue, as described in more detail below.

The cutting edge of cutting instruments suitable for use in the methods of the present invention can comprise any medically acceptable material. In some preferred embodiments, the cutting edge of cutting instruments suitable for use in the methods of the present invention comprises a metal (e.g., a stainless steel). Non-limiting examples of cutting instruments suitable for use in the methods of the present invention are described in U.S. Pat. No. 7,037,175 to Spiro et al., which is incorporated herein by reference. In other embodiments, the cutting edge comprises a ceramic or metal oxide material.

The methods of the present invention provide one or more of the following benefits to a patient recovering from a surgical procedure compared to surgery with a conventional surgical blade: reduced post-operative pain, reduced post-operative swelling, more rapid wound closure, reduced inflammation, better and more rapid cell reorganization around the incision, reduced scarring, higher tissue strength for the healed or healing incision, reduced morbidity of tissue at the site of the surgical incision, and faster reepithelialization of the incision site.

In another aspect, the present invention provides improved cutting instruments in which the ultimate cutting edge and/or each beveled face adjacent the ultimate edge of the cutting instrument has a maximum height deviation of about 4 μm or less along any 680 μm long segment thereof and each beveled face adjacent the ultimate edge has a RMS surface roughness of about 200 nm or less.

In yet another aspect, the present invention provides a method for reducing batch-to-batch variability in the manufacture of cutting instruments, as described in more detail below.

### BRIEF DESCRIPTION OF THE DRAWINGS

FIG. 1 shows a side view of a cutting instrument for use in accordance with the methods of the invention.

FIG. 2 is a schematic drawing illustrating the method used to characterize the contour of the ultimate edge of a cutting instrument.

FIG. 3 shows a photomicrograph of a commercial BARD-PARKER® No. 15 surgical blade, as received, at a magnification of about 450×.

FIG. 4 shows a photomicrograph ff a BARD-PARKER® No. 15 surgical blade, polished according to the invention, at a magnification of about 450×.

### DETAILED DESCRIPTION OF THE INVENTION

The present invention provides methods for ameliorating tissue trauma from surgical incisions and promoting healing of surgically incised tissues.

A first aspect of the present invention is a method for ameliorating tissue trauma (e.g., tearing, cell surface damage, and the like) from a surgical incision. The method comprises making the surgical incision with a highly polished cutting instrument that preferably comprises a cutting instrument body having two opposed sides and a direction of elongation, and includes at least one cutting edge extending along the direction of elongation. The at least one cutting edge defines an ultimate edge and two beveled faces adjacent the ultimate edge. The surfaces of the cutting edge of the cutting instrument are smooth and even, and have at least one of the following characteristics: (a) the ultimate edge has a maximum height deviation of about 4 μm or less along any 680 μm long segment thereof; (b) each beveled face adjacent the ultimate edge has a maximum height deviation of about 4 μm or less along any 680 μm long linear segment thereof; and (c) each beveled face adjacent the ultimate edge has a root mean square (RMS) surface roughness (Rq) of about 200 nm or less, as determined by surface metrology techniques that are well known in the art. The smoothness of the sides of the highly polished cutting instrument and the evenness of the cutting edge provide for cleaner cuts and reduced snagging and tearing of tissue.

A second aspect of the present invention is a method for promoting healing of surgically incised tissue. The method comprises making a surgical incision with a highly polished cutting instrument that preferably comprises a cutting instrument body having two opposed sides and a direction of elongation, and includes at least one cutting edge extending along the direction of elongation. The at least one cutting edge defines an ultimate edge and two beveled faces adjacent the ultimate edge. The surfaces of the cutting edge of the cutting instrument are smooth and even, and have at least one of the following characteristics: (a) the ultimate edge has a maximum height deviation of about 4 μm or less along any 680 μm long segment thereof; (b) each beveled face adjacent the ultimate edge has a maximum height deviation of about 4 μm or less along any 680 μm long linear segment thereof; and (c) each beveled face adjacent the ultimate edge has a RMS surface roughness of about 200 nm or less. It is believed that cleaner cuts afforded by the present method enhances and promotes healing by providing cut tissue surfaces with less damage, thereby providing a sufficient level of tissue trauma to initiate healing during the inflammatory phase of the healing process, without over stimulating the immune system and other reparative mechanisms. Over stimulation of the body's reparative mechanisms can lead to undesirably high levels of inflammation, swelling, and the like, which can impede the healing process.

A third aspect of the present invention is a method for ameliorating scarring of surgically incised tissue. The method comprises making a surgical incision with a highly polished cutting instrument that preferably comprises a cutting instrument body having two opposed sides and a direction of elongation, and includes at least one cutting edge extending along the direction of elongation. The at least one cutting edge defines an ultimate edge and two beveled faces adjacent the ultimate edge. The surfaces of the cutting edge of the cutting instrument are smooth and even, and have at least one of the following characteristics: (a) the ultimate edge has a maximum height deviation of about 4 μm or less along any 680 μm long segment thereof; (b) each beveled face adjacent the ultimate edge has a maximum height deviation of about 4 μm or less along any 680 μm long linear segment thereof; and (c) each beveled face adjacent the ultimate edge has a RMS surface roughness of about 200 nm or less. The present method provides reduced scarring of surgically incised tissue after the healing process is complete. In part, this effect may be due to the more even deposition of collagen observed during the proliferation and maturation phases of the healing process in tissues incised with the highly polished cutting instruments of the invention, compared to tissues incised with conventional surgical blades. Scarring is also associated with excessive inflammation at wound sites. The reduced scarring provided by the present method may be due, in part, to the lower level of inflammation that occurs in tissue incised with the highly polished cutting instruments of the invention compared to conventional surgical blades.

A fourth aspect of the present invention is a method for ameliorating inflammation of surgically incised tissue. The method comprises making a surgical incision with a highly polished cutting instrument that preferably comprises a cutting instrument body having two opposed sides and a direction of elongation, and includes at least one cutting edge extending along the direction of elongation. The at least one cutting edge defines an ultimate edge and two beveled faces adjacent the ultimate edge. The surfaces of the cutting edge of the cutting instrument are smooth and even, and have at least one of the following characteristics: (a) the ultimate edge has a maximum height deviation of about 4 μm or less along any 680 μm long segment thereof; (b) each beveled face adjacent the ultimate edge has a maximum height deviation of about 4 μm or less along any 680 μm long linear segment thereof; and (c) each beveled face adjacent the ultimate edge has a RMS surface roughness of about 200 nm or less. As noted above, incisions made with the highly polished cutting instruments of the invention provide a sufficient level of tissue trauma to initiate the healing process, without over stimulating the reparative mechanisms that come into play during wound healing. This results in reduced levels of inflammation compared to incisions made with conventional surgical blades.

A fifth aspect of the present invention is a method for promoting closure of surgically incised tissue. The method comprises making a surgical incision with a highly polished cutting instrument that preferably comprises a cutting instrument body having two opposed sides and a direction of elongation, and includes at least one cutting edge extending along the direction of elongation. The at least one cutting edge defines an ultimate edge and two beveled faces adjacent the ultimate edge. The surfaces of the cutting edge of the cutting instrument are smooth and even, and have at least one of the following characteristics: (a) the ultimate edge has a maximum height deviation of about 4 μm or less along any 680 μm long segment thereof; (b) each beveled face adjacent the ultimate edge has a maximum height deviation of about 4 μm or less along any 680 μm long linear segment thereof; and (c) each beveled face adjacent the ultimate edge has a RMS surface roughness of about 200 nm or less. More rapid closure of the surgically incised tissue may result, at least in part, due to the reduced levels of swelling and inflammation and more uniform collagen deposition, which occur in tissue incised with the highly polished cutting instruments of the invention, compared to conventional surgical blades. The more rapid closure provided by the present method can beneficially reduce the risk of wound contamination and infection, by reducing the time during which foreign matter can potentially enter the incision site.

A sixth aspect of the present invention is a method for promoting tissue strength in a healing and/or healed surgically incised tissue. The method comprises making a surgical incision with a highly polished cutting instrument that preferably comprises a cutting instrument body having two opposed sides and a direction of elongation, and includes at least one cutting edge extending along the direction of elongation. The at least one cutting edge defines an ultimate edge and two beveled faces adjacent the ultimate edge. The surfaces of the cutting edge of the cutting instrument are smooth and even, and have at least one of the following characteristics: (a) the ultimate edge has a maximum height deviation of about 4 μm or less along any 680 μm long segment thereof; (b) each beveled face adjacent the ultimate edge has a maximum height deviation of about 4 μm or less along any 680 μm long linear segment thereof; and (c) each beveled face adjacent the ultimate edge has a RMS surface roughness of about 200 nm or less.

The present method can provide an ultimate healed-tissue strength almost equal to virgin (uncut) tissue. Good tissue strength in healed incisions is of great importance to many individuals, particularly athletes who have undergone joint surgery, and wish to continue their athletic pursuits. Weak tissue is more prone to subsequent injury and can be painful when stressed, compared to the relatively strong healed tissue provided by the present methods. In addition, the present methods can provide for stronger tissue during the healing process, which advantageously lessens the likelihood that the incision will reopen or tear when stressed (e.g., by movement and exercise).

A seventh aspect of the present invention is a method for promoting reepithelialization of surgically incised tissue. The method comprises making a surgical incision with a highly polished cutting instrument that preferably comprises a cutting instrument body having two opposed sides and a direction of elongation, and includes at least one cutting edge extending along the direction of elongation. The at least one cutting edge defines an ultimate edge and two beveled faces adjacent the ultimate edge. The surfaces of the cutting edge of the cutting instrument are smooth and even, and have at least one of the following characteristics: (a) the ultimate edge has a maximum height deviation of about 4 μm or less along any 680 μm long segment thereof; (b) each beveled face adjacent the ultimate edge has a maximum height deviation of about 4 μm or less along any 680 μm long linear segment thereof; and (c) each beveled face adjacent the ultimate edge has a RMS surface roughness of about 200 nm or less. Reepithelialization is the process whereby epithelial cells associate at the wound site and form a contiguous epithelial tissue. Efficient wound closure of epithelial tissues (e.g., skin) is important for restoring the barrier function of such tissues (e.g., to prevent infection and the like). Accordingly, the rapid reepithelialization afforded by making surgical incisions with the highly polished cutting instruments of the present invention can beneficially reduce the potential for infection and contamination of the wound site.

An eighth aspect of the present invention is a method for ameliorating swelling during healing of surgically incised tissue. The method comprises making a surgical incision with a highly polished cutting instrument that preferably comprises a cutting instrument body having two opposed sides and a direction of elongation, and includes at least one cutting edge extending along the direction of elongation. The at least one cutting edge defines an ultimate edge and two beveled faces adjacent the ultimate edge. The surfaces of the cutting edge of the cutting instrument are smooth and even, and have at least one of the following characteristics: (a) the ultimate edge has a maximum height deviation of about 4 μm or less along any 680 μm long segment thereof; (b) each beveled face adjacent the ultimate edge has a maximum height deviation of about 4 μm or less along any 680 μm long linear segment thereof; and (c) each beveled face adjacent the ultimate edge has a RMS surface roughness of about 200 nm or less. The reduced swelling during healing of tissue that has been surgically incised by the highly polished cutting instruments of the present invention can beneficially reduce the level of pain often associated with the healing process, as well as reduce the stress on the healing tissue.

A ninth aspect of the present invention is a method for ameliorating tissue morbidity during the healing of surgically incised tissue. The method comprises making a surgical incision with a highly polished cutting instrument that preferably comprises a cutting instrument body having two opposed sides and a direction of elongation, and includes at least one cutting edge extending along the direction of elongation. The at least one cutting edge defines an ultimate edge and two beveled faces adjacent the ultimate edge. The surfaces of the cutting edge of the cutting instrument are smooth and even, and have at least one of the following characteristics: (a) the ultimate edge has a maximum height deviation of about 4 μm or less along any 680 μm long segment thereof; (b) each beveled face adjacent the ultimate edge has a maximum height deviation of about 4 μm or less along any 680 μm long linear segment thereof; and (c) each beveled face adjacent the ultimate edge has a RMS surface roughness of about 200 nm or less. Tissue morbidity is an undesirable side-effect of tissue trauma. In extreme cases, tissue can be so damaged as to simply not heal, leading to open, suppurating wounds, which can lead to systemic infections and/or the need to surgically excise the damaged tissue or even amputate an affected appendage. The reduced trauma to tissue that has been surgically incised with a highly polished cutting instrument of the present invention provides the added benefit of lower morbidity compared to incisions made with conventional surgical blades.

A tenth aspect of the present invention is a highly polished cutting instrument comprising a cutting instrument body having two opposed sides and a direction of elongation, and including at least one cutting edge extending along the direction of elongation and defining an ultimate edge and two beveled faces adjacent the ultimate edge. The ultimate edge and/or the beveled faces adjacent the ultimate edge have height deviation of not more than about 4 μm along any 680 μm long segment thereof, and each beveled face adjacent the ultimate edge has a RMS surface roughness of not more than about 200 nm. The cutting instruments of the present invention are particularly suited for use in surgical procedures, providing surgical incisions that are cleaner and less damaging to tissue than conventional surgical blades. The cleanness and smoothness of the incisions made with the cutting instruments of the invention leads to one or more benefits, including, without limitation, reduced swelling during healing, more rapid wound closure, reduced tendency toward infection, reduced trauma to the incised tissue, more rapid reepithelialization of the incised tissue, and the like, as described in detail herein.

An eleventh aspect of the present invention is a method of reducing batch-to-batch variability in cutting instrument manufacture comprising polishing the cutting edge of each cutting instrument in each batch of cutting instruments in a manufacturing run to provide a cutting edge for each instrument in each batch, in which the ultimate edge and/or each beveled face adjacent the ultimate edge has a maximum height deviation of not more than about 4 μm along any 680 μm long segment thereof; and each beveled face adjacent the ultimate edge has a RMS surface roughness of not more than about 200 nm. Preferably, the polishing of the cutting edge is accomplished by buffing the surfaces of the cutting edge to rapidly remove uneven material from the surfaces, and optionally chemically-mechanically polishing the buffed surfaces to provide the desired level of smoothness and minimize height deviations. The surfaces of the resulting highly polished cutting instruments are then cleaned to remove any debris left over from the polishing process. For surgical applications, the cutting instruments are also preferably sterilized after cleaning, and then packaged in sterile packaging materials for distribution and sale.

In preferred embodiments of the present cutting instruments and methods, the maximum height deviation along any 680 μm long segment of the ultimate edge and/or the beveled faces of the cutting edge is about 1 μm or less. Preferably, the RMS surface roughness of the beveled faces adjacent the ultimate edge are in the range of about 10 nm to about 150 nm, more preferably about 20 nm to about 80 nm. In some preferred embodiments, the RMS high band-pass edge roughness (Rq) of the beveled edges is not more than about 50 nm, preferably, the high band-pass Rq is in the range of about 1 nm to about 30 nm, more preferably, the high band-pass Rq is in the range of about 1 nm to about 10 nm.

Examples of cutting instruments suitable for use in the present methods include, but are not limited to, knives, surgical scalpels, scissors, and razors used to cut living tissue. The cutting instrument can have any suitable additional features, for example, a separate handle attached to the cutting instrument by suitable means. Surgical blades can be configured in a variety of ways depending on the intended use of the blade, as is well known in the art. For example, the cutting edge can be curved or straight. Cutting edges can curve in plane with the direction of elongation, or in some instances can also or alternatively curve at an angle from the direction of elongation (i.e., to form a scoop-shaped blade). Blades of any surgical configuration, which have the proper surface roughness and or edge regularity, can be used in the methods of the present invention. Examples of different cutting instrument configurations that can be used in the present methods include fitment blades, in which the blade is separate from the handle and is removably attached thereto, disposable or reusable scalpels having curved or straight cutting edges, scalpels having retractable blades, stitch cutters, which have a concave curvature along the cutting edge, skin graft blades, which fit Braithwaite, Cobbett, and Watson knives, cervical biopsy blades, which have two cutting edges that meet in a point at the foremost tip of the blade, endoscopic scissors, lancets, tissue cutters (e.g., aortic punches, biopsy punches), dissecting blades, cutting forceps, harmonic scalpels used with ultrasound devices, surgical curvets (e.g., scoops type blades), cataract blades, and the like. Surgical blades that can be highly polished as described herein for use in the present invention are available from a number of suppliers, including, without limitation, Swann-Morton Limited of Sheffield, England; and Becton, Dickenson Company of Franklin Lakes, N.J., USA (e.g., Bard-Parker blades, Beaver blades).

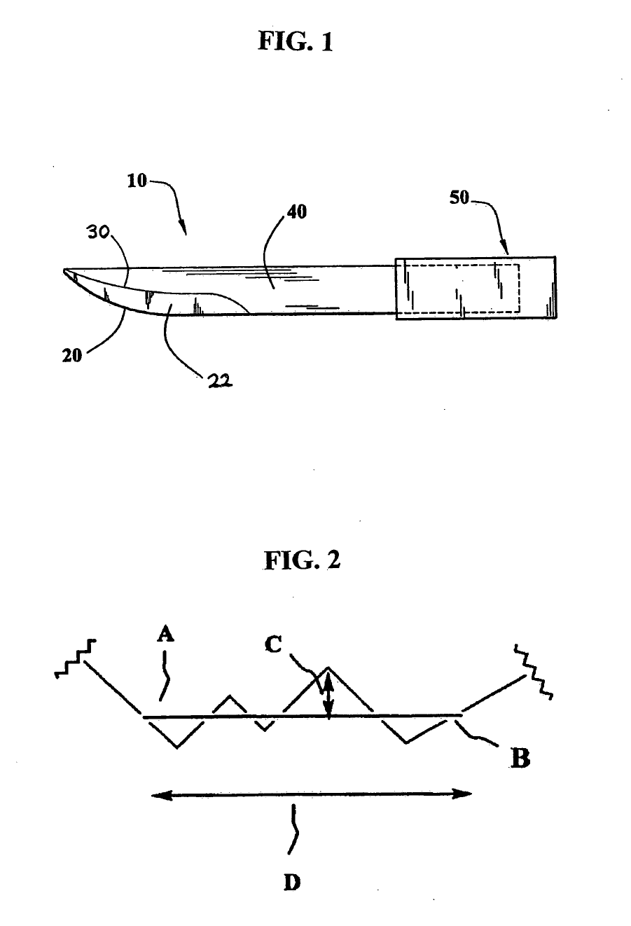

FIG. 1 shows a side view of a cutting instrument (10) suitable for use in the methods of the present invention. In general any cutting instrument has a body (40) and a cutting edge (30). In such cutting instruments the cutting edge is defined as that portion of the cutting instrument having beveled faces (22) which taper to a terminating, or ultimate edge (20). The body (40) of the cutting instrument is defined as the structure that transfers an applied load from the cutting instrument driving force to the ultimate edge (20) of the cutting edge (30). In addition, as shown in FIG. 1, a cutting instrument can include an optional handle or grip (50) which serves as a stable interface between the cutting instrument user and the cutting instrument. Cutting edge (30) of the cutting instrument is characterized by a uniform edge having a maximum height deviation of not more than about 4 μm along any 680 μm long segment thereof, and/or uniform beveled faces adjacent the ultimate cutting edge having a maximum height deviation of not more than about 4 μm along any 680 μm long linear segment of the faces, and/or beveled faces adjacent the ultimate edge having a RMS surface roughness of not more than about 200 nm.

The cutting edge can be integral with the cutting instrument body and can be formed directly on a cutting instrument body, thus comprising the same material as the cutting instrument body. Alternatively, the cutting edge can be non-integral with the cutting instrument body, and can be formed by layering or otherwise attaching a second material on the first material of the cutting instrument body. In addition, the cutting edge can have a convex curvature, as shown in FIG. 1, can be straight, can have a concave curvature, or can have a complex curvature including portions that have concave curvature, portions that have convex curvature, portions that are straight, portions that have scoop-like shape, or any combination thereof.

The cutting instrument and cutting edge can comprise any suitable material. In some embodiments, the cutting instrument and cutting edge each comprises a metal. For example, the metal can be a metal alloy, e.g., an alloy of iron with at least one element selected from the group consisting of carbon, chromium, nickel, and cobalt. Preferred alloys of iron include stainless steels and carbon steels. The cutting instrument and cutting edge can comprise any stainless steel or carbon steel. Stainless steels are typically comprised of iron and chromium in various proportions, although other elements, including silicon, nickel, molybdenum, phosphorus, sulfur, copper, and aluminum, are often components of commercially available stainless steels. Carbon steels typically comprise iron and carbon, and can include additional elements, including chromium, nickel molybdenum, vanadium, and silicon. Carbon steels are typically further classified as mild steels, comprising less than about 0.25 wt. % carbon, medium carbon steels, comprising about 0.25 wt. % to 0.45 wt. % carbon, and high carbon steels, comprising about 0.45 wt. % to 1.5 wt. % carbon.

The metal can be any suitable bulk amorphous alloy. Generally, bulk amorphous alloys are formed by solidification of alloy melts by cooling the alloy to a temperature below its glass transition temperature before appreciable homogeneous nucleation and crystallization has occurred. Many bulk amorphous alloys are known in the art, all of which are suitable for use in the cutting instrument of the invention.

In other embodiments, the cutting instrument comprises a metal oxide material such as sapphire (aluminum oxide) or a ceramic material, or a any combination of a metal, a metal oxide, and/or a ceramic material, for example.

The cutting edge can be formed by any suitable method. For example, the cutting edge can be polished by the methods disclosed in U.S. Pat. No. 7,037,175 to Spiro et al. Conventional methods of grinding, honing, and stropping can be used to prepare a cutting edge before polishing, if desired.

In the methods of Spiro et al. (U.S. Pat. No. 7,037,175), the cutting edge is contacted with a polishing pad and a chemical-mechanical polishing composition. The polishing surface of the polishing pad can comprise any suitable material, many of which are known in the art. Suitable polishing pads include, for example, woven and non-woven polishing pads. Moreover, suitable polishing pads can comprise any suitable polymer of varying density, hardness, thickness, compressibility, ability to rebound upon compression, and compression modulus. Suitable polymers include, for example, polyvinylchloride, polyvinylfluoride, nylon, fluorocarbon, polycarbonate, polyester, polyacrylate, polyether, polyethylene, polyamide, polyurethane, polystyrene, polypropylene, coformed products thereof, and mixtures thereof.

The polishing pad can have any suitable configuration. For example, the polishing pad can be circular and when in use have a rotational motion about an axis perpendicular to the plane defined by the surface of the pad. The polishing pad can be cylindrical, the surface of which acts as the polishing surface, and when in use have a rotational motion about the central axis of the cylinder. The polishing pad can be in the form of an endless belt, which when in use has a linear motion with respect to the cutting edge being polished. The polishing pad can have any suitable shape, and when in use have a reciprocating or orbital motion along a plane or a semicircle. Many other variations will be readily apparent to the skilled artisan.

The chemical-mechanical polishing composition for polishing the cutting edge of a cutting instrument for use in the present methods comprises particles of an abrasive and a liquid carrier, wherein the abrasive is suspended in the liquid carrier. The abrasive can be any suitable abrasive and preferably is selected from the group consisting of silica, alumina, ceria, titania, zirconia, germania, diamond, polycarbonate, silicon carbide, titanium carbide, titanium nitride, niobium carbide, chromium carbide, and mixtures thereof. More preferably, the abrasive is silica. The cutting edge can be buffed prior to CMP, if desired. For example, the surface can be buffed with a chromium oxide-containing buffing composition and the like prior to CMP.

The silica can be any suitable form of silica. Suitable forms of silica include fumed silica and colloidal silica. Fumed silica is typically prepared by a pyrogenic process, in which a suitable precursor, such as silicon tetrachloride, undergoes vapor phase hydrolysis at high temperatures. Colloidal silica useful in the context of the invention includes wet-process type silica particles (e.g., condensation-polymerized silica particles). Condensation-polymerized silica particles typically are prepared by condensing Si(OH)₄ to form colloidal particles, where colloidal is defined as having an average particle size between 1 nm and 1000 nm. Such abrasive particles can be prepared in accordance with U.S. Pat. No. 5,230,833 or can be obtained as any of various commercially available products, such as the Akzo-Nobel BINDZIL® 50/80 product and the Nalco 1050, 2327, and 2329 products, as well as other similar products available from DuPont, Bayer, Applied Research, Nissan Chemical, and Clariant.

The abrasive particles typically have an average particle size (e.g., average particle diameter) of 20 nm to 500 nm. Preferably, the abrasive particles have an average particle size of 70 nm to 300 nm (e.g., 100 nm to 200 nm).

The abrasive can be present in any suitable amount. Typically, 0.001 wt. % or more abrasive (e.g., 0.01 wt. % or more) can be present in the polishing composition. The amount of abrasive in the polishing composition preferably will not exceed 40 wt. %, and more preferably will not exceed 20 wt. % (e.g., will not exceed 10 wt. %). Even more preferably, the amount of the abrasive will be 0.01 wt. % to 10 wt. % of the polishing composition.

The abrasive is suspended in the polishing composition, more specifically in the liquid carrier of the polishing composition. The abrasive preferably is colloidally stable. The term colloid refers to the suspension of abrasive particles in the liquid carrier. Colloidal stability refers to the maintenance of that suspension over time. In the context of this invention, an abrasive is considered colloidally stable if, when the abrasive is placed into a 100 ml graduated cylinder and allowed to stand unagitated for a time of 2 hours, the difference between the concentration of particles in the bottom 50 ml of the graduated cylinder ([B] in terms of g/ml) and the concentration of particles in the top 50 ml of the graduated cylinder ([T] in terms of g/ml) divided by the initial concentration of particles in the abrasive composition ([C] in terms of g/ml) is less than or equal to 0.5, i.e., ([B]−[T])/[C]≧0.5. The value of ([B]−[T])/[C] desirably is less than or equal to 0.3, and preferably is less than or equal to 0.1.

The chemical-mechanical polishing composition optionally can comprise an oxidizing agent. Without wishing to be bound by any particular theory, it is believed that the oxidizing agent reacts with the surface of the cutting edge to form a soft oxidized film that is easily abraded by suspended abrasive particles. The oxidizing agent can be any oxidizing agent capable of oxidizing the material from which the cutting edge of the cutting instrument is formed. Preferably, the oxidizing agent is selected from the group consisting of bromates, bromites, chlorates, chlorites, ferric nitrate, hydrogen peroxide, hypochlorites, iodates, monoperoxy sulfate, monoperoxy sulfite, monoperoxyphosphate, monoperoxyhypophosphate, monoperoxypyrophosphate, organo-halo-oxy compounds, periodates, permanganate, and peroxyacetic acid. A preferred example of an oxidizing agent is hydrogen peroxide. As will be appreciated by one of ordinary skill in the art, the choice of the oxidizing agent will depend on the material comprising the cutting edge.

The polishing composition can comprise any suitable amount of the oxidizing agent. Typically, the polishing composition comprises 0.1 wt. % or more (e.g., 0.2 wt. % or more, 0.5 wt. % or more, or 1 wt. % or more) oxidizing agent, based on the weight of the liquid carrier and any components dissolved or suspended therein. The polishing composition preferably comprises 20 wt. % or less (e.g., 15 wt. % or less, or 10 wt. % or less) oxidizing agent, based on the weight of the liquid carrier and any components dissolved or suspended therein.

The liquid carrier can be any suitable liquid carrier. The purpose of the liquid carrier is to facilitate the application of the components of the polishing composition to the substrate surface to be polished. Typically, the liquid carrier is water, a mixture of water and a suitable water-miscible solvent, or an emulsion. Preferably, the liquid carrier comprises, consists essentially of, or consists of water, more preferably deionized water.

The chemical-mechanical polishing composition can have any suitable pH. Typically, the polishing composition will have a pH of 12 or less (e.g., 11 or less, or 10 or less). Preferably, the polishing composition will have a pH of 1 or more (e.g., 2 or more, or 3 or more).

The pH of the polishing composition can be achieved and/or maintained by any suitable means. More specifically, the polishing composition can further comprise a pH adjustor, a pH buffering agent, or a combination thereof. The pH adjustor can be any suitable pH-adjusting compound. For example, the pH adjustor can be a base such as potassium hydroxide, sodium hydroxide, ammonium hydroxide, or a combination thereof. Alternatively, the pH adjusting agent can be an acid (e.g., hydrochloric acid, sulfuric acid, and the like), or a combination of an acid and a base, as needed. The pH buffering agent can be any suitable buffering agent, for example, phosphates, acetates, borates, ammonium salts, and the like. The chemical-mechanical polishing composition can comprise any suitable amount of a pH adjustor and/or a pH buffering agent, provided such amount is sufficient to achieve and/or maintain the pH of the polishing system within the ranges set forth herein.

The chemical-mechanical polishing composition optionally can further comprise one or more other additives. Such additives include any suitable surfactant and/or rheological control agent, including viscosity enhancing agents and coagulants (e.g., polymeric rheological control agents, such as, for example, urethane polymers), acrylates comprising one or more acrylic subunits (e.g., vinyl acrylates and styrene acrylates), and polymers, copolymers, and oligomers thereof; and salts thereof. Suitable surfactants include, for example, cationic surfactants, anionic surfactants, anionic polyelectrolytes, nonionic surfactants, amphoteric surfactants, fluorinated surfactants, mixtures thereof; and the like.

The chemical-mechanical polishing composition optionally further comprises an antifoaming agent. The anti-foaming agent can be any suitable anti-foaming agent. Suitable antifoaming agents include, but are not limited to, silicon-based and acetylenic diol-based antifoaming agents. The amount of anti-foaming agent present in the polishing composition typically is 40 ppm to 140 ppm.

The chemical-mechanical polishing composition optionally can further comprise a biocide. The biocide can be any suitable biocide, for example an isothiazolinone biocide. The amount of biocide used in the polishing composition typically is 1 to 50 ppm, preferably 10 to 20 ppm.

In preparing a cutting instrument suitable for use in the present invention, the cutting edge can be sharpened and/or highly polished by any suitable technique. In a preferred method the cutting edge is highly polished until the cutting edge is uniform and has a maximum height deviation of not more than about 4 μm along any 680 μm segment of the ultimate edge, and/or a maximum height deviation of not more than about 4 μm along any 680 μm linear segment of each beveled face adjacent the ultimate edge, and/or a RMS surface roughness of not more than about 200 nm on each beveled face adjacent the ultimate edge.

Preferably, the cutting edge is chemically-mechanically polished, typically by pressing a polishing pad against the cutting edge at an angle, in the presence of a polishing composition under controlled chemical, pressure, velocity, and temperature conditions. The preferred method of chemically-mechanically polishing the cutting edge is particularly suited for use in conjunction with a chemical-mechanical polishing (CMP) apparatus. Typically, the apparatus comprises a platen, which, when in use, is in motion and has a velocity that results from orbital, linear, or circular motion, a polishing pad in contact with the platen and moving with the platen when in motion, and a carrier that holds a substrate to be polished by contacting and moving relative to the surface of the polishing pad. The cutting instrument having a cutting edge can be mounted in a carrier that is adjustable with respect to the angle at which the cutting edge contacts the polishing pad. The polishing of the substrate takes place by the cutting edge being placed in contact with the polishing pad and the polishing composition of the invention and then the polishing pad moving relative to the cutting edge (with the polishing composition therebetween), so as to abrade at least a portion of the cutting edge to polish the cutting edge.

The chemical-mechanical polishing composition can be formulated prior to delivery to the polishing pad or to the surface of the cutting edge. The polishing composition can also be formulated (e.g., mixed) on the surface of the polishing pad or on the surface of the cutting edge, through delivery of the components of the polishing composition from two or more distinct sources, whereby the components of the polishing composition meet at the surface of the polishing pad or at the surface of the cutting edge. In this regard, the flow rate at which the components of the polishing composition are delivered to the polishing pad or to the surface of the cutting edge (i.e., the delivered amount of the particular components of the polishing composition) can be altered prior to the polishing process and/or during the polishing process, such that the polishing selectivity and/or viscosity of the polishing composition is altered. Moreover, it is suitable for the particular components of the polishing composition being delivered from two or more distinct sources to have different pH values, or alternatively to have substantially similar, or even equal, pH values, prior to delivery to the surface of the polishing pad or to the surface of the cutting edge. It is also suitable for the particular components being delivered from two or more distinct sources to be filtered either independently or to be filtered jointly (e.g., together) prior to delivery to the surface of the polishing pad or to the surface of the cutting edge.

In addition, the cutting edge can be finished with a coating, if desired, e.g., a protective and/or strengthening coating after polishing the cutting edge. A protective and/or friction reducing layer of, for example, polytetrafluoroethylene, silicones, polyethylene, etc., can be applied to the cutting edge after the polishing operation. Strengthening coatings can be applied, as well. A non-limiting example of a strengthening coating comprises a coating formed by application to the cutting edge of a molybdenum layer as a diffusion barrier, followed by deposition of diamond-like carbon. Another example of a strengthening coating is titanium nitride. Other examples of post-polishing coatings will be readily apparent to those skilled in the art.

The methods of the present inventive utilize cutting instruments having an extremely even and smooth cutting edge. Typically, scanning electron microscopy of conventional cutting edges shows that, when examined at an angle perpendicular to the plane defined by the cutting instrument body upon which is formed the cutting edge, the ultimate edge of the cutting edge comprises an uneven contour. Points along the ultimate edge of the cutting edge will have a deviation from a line defined by two points on the ultimate edge of the cutting edge. The magnitude of the deviation will typically increase as the distance between the two points of the ultimate edge of the cutting edge defining the line used as a reference for the measurement increases. With reference to FIG. 2, the parameter used to characterize the contour of the ultimate edge of the cutting edge is the deviation between (a) a line D drawn between two points A, B on the ultimate edge and (b) a point C on the actual ultimate edge between the aforesaid two points A, B. Conventional cutting instruments have an ultimate edge that typically will have a minimum deviation from a line defined by two points on the ultimate edge separated by 680 μm of any point on the ultimate edge between the two points of 5 μm or greater. Similarly, for conventional cutting edge instruments, the minimum deviation from a line defined by two points on the ultimate edge separated by 680 μm of any point on the ultimate edge between the two points is 2 μm or greater and/or the minimum deviation from a line defined by two points on the ultimate edge separated by 10 μm of any point on the ultimate edge between the two points is 1.5 μm or greater.

For use in the present invention, a cutting edge of a cutting instrument shows a significantly more uniform or even contour of the ultimate edge when examined by scanning electron microscopy as discussed above than cutting edges produced by conventional practices. Typically, a cutting edge formed along a cutting instrument body, wherein the cutting instrument body comprises an alloy of iron with at least one element selected from the group consisting of carbon, chromium, nickel, and cobalt, suitable for use in the present methods, will have a maximum height deviation along any 680 μm segment of the ultimate edge that is not more than about 4 μm (i.e., will have a maximum height deviation from any line defined by two points on the ultimate edge separated by 680 μm, for any point on the ultimate edge between the two points, of about 4 μm or less, e.g., 3.5 μm or less, or 3 μm or less, or even 2.5 μm or less). The maximum height deviation along any 680 μm segment of the ultimate edge preferably is not more than about 1 μm (e.g., 0.9 μm or less, or 0.8 μm or less, or even 0.7 μm or less), and the maximum height deviation from a line defined by two points on the ultimate edge separated by 10 μm of any point on the ultimate edge between the two points is 0.5 μm or less (e.g., 0.4 μm or less, or 0.3 μm or less).

It is particularly preferred that a cutting instrument utilized in the present methods has a cutting edge (30) as shown in FIG. 1 having beveled faces (22) adjacent the ultimate edge with significantly reduced surface roughness than conventional surgical blades. Surface roughness is a measure of the depth of surface variations and a number of methods are well known in the art to determine surface roughness. The surface roughness can be measured mechanically by moving a stylus along a surface or by using light scattering techniques. The American Society of Mechanical Engineers (ASME) standard B46.1-2002 contains descriptions of methods used to measure and express surface roughness. As discussed above, preferably, the RMS surface roughness (Rq) of each beveled face adjacent the ultimate edge of the cutting instrument is not more than about 200 nm.

The amelioration of tissue trauma and wound healing promotion provided by the methods of the present invention can be observed and quantified by methods that are well known in the art. For example, incisions made with sharpened blades according to the present invention can close faster than incisions made with conventional surgical blades. In addition, less post-operative inflammation, less collagen deposition, reduced scarring, and improved tissue strength can be observed with incisions made according to the present methods compared to incisions made with conventional surgical blades.

Wound healing and reduction in tissue trauma can be assessed by any suitable animal model (e.g., guinea pigs, mice, rats, swine, or other mammals). For example, surgical incisions can be made in an animal, such as a guinea pig, under standard surgical protocols, using conventional surgical scalpels and ultrapolished (UP) scalpels according to the present invention. The incisions can be closed by any suitable method, such as by suture or staple. The closed incisions are treated and dressed post-operatively under standard and customary conditions well known in the medical art for care of surgical incisions. The rapidity of incision healing and the extent of tissue trauma can be assessed for each incision using standard histochemical, microscopic, and other known techniques. For example, the degree of post-operative inflammation can be assessed by immunohistochemical tests and the like, the degree of collagen deposition can be assessed by various microscopic staining techniques and the like, the evenness and rate of wound closure can be assessed microscopically and visually, and the degree of scarring can be assessed microscopically and visually, using standard techniques that are well known in the medical arts. Tissue strength in the healed wound can also be assessed by standard methods.

Incisions made according to the methods of the present invention can result in one or more of the following benefits, including, without limitation, reduced post-operative inflammation, reduced post-operative swelling, reduced post-operative infection, reduced or more ordered collagen deposition, more even tissue healing, reduced or more even scarring, and more rapid healing of the incision, as compared to incisions made using conventional surgical blades.

The methods of the present invention provide a number of potential benefits to a patient recovering from a surgical procedure compared to surgery with a conventional surgical blade. For example, post-operative pain may be reduced due to more rapid wound closure and healing, reduced inflammation, and reduced post-operative swelling. The methods of the invention provide better and more rapid cell reorganization around the incision, reduced scarring, higher tissue strength for the healed or healing incision, and faster reepithelialization of the incisions. Improved tissue strength can be particularly important for patients that must undergo a number of surgical procedures in the same surgical field over a period of time. For example, women who have multiple caesarian births often experience weakening of the uterus, causing a potential for uterine rupture, which can limit the number of pregnancies such women can safely undergo. A stronger healed incision could benefit such women, allowing a greater number of safe pregnancies, for example.

### Example 1

Surgical blades suitable for use in the methods of the present invention were prepared by polishing commercially available BARD-PARKER® No. 15 stainless-steel surgical scalpels (Becton Dickenson AcuteCare, Franklin Lakes, N.J.) according to the procedures described below. Sets of scalpels were polished by buffing with chromium oxide buffing compound in a single pass, as well as in multiples passes. In addition, blades were polished by first buffing with chromium oxide, followed by chemical-mechanical polishing. The surface roughness of the blades was evaluated using “cylinder and tilt” metrology to determine the average surface roughness (Ra), the RMS surface roughness (Rq), and peak to valley roughness (Rz), which is determined using the 5 highest and 5 lowest points on the surface, as well as by high band-pass metrology, all of which are well known in the art. Comparison was made to commercial blades, as received, including diamond blades (CVD Diamond Knife for Soft Tissue, Scalpel No. 15, from Rhein Medical, Inc., Tampa, Fla.), BD Beaver mini-blades (BD Ophthalmic Systems, Waltham, Mass.), BARD-PARKER® No. 15 stainless-steel surgical blades, and IONFusion stainless steel, single use surgical scalpel blades, size 15 (Cat. No. 100-015, IonFusion Surgical, El Cajon, Calif.). The cutting instruments of the invention were polished by a one-pass buffing process, a multi-pass buffing process, or a combination of buffing and chemical-mechanical polishing.

The buffing processes involved buffing the cutting surfaces of the blades on a 6 inch by ¾ inch, hard felt buffing wheel loaded with tricyclo chromium oxide (Cr₂O₃) buffing compound at about 3450 revolutions per minute (rpm). Each blade was mounted in a jig and a cutting surface of the blade was pressed against the moving wheel until it flexed (about 1 mm). The blade was then slowly moved across the rotating wheel, following the contour of the cutting surface of the blade (about 1 second per pass). The blade was then removed from the jig, and remounted with the other side of the blade directed toward the buffing wheel and buffed as described above on that side. The blade was then cleaned with a tissue wetted with a 10 percent solution of isopropanol in water to remove debris remaining from the buffing process. In the multi-pass buffing process, each side was buffed as described a total of 6 times.

Certain of the multi-pass-buffed blades were also chemically-mechanically polished (CMP) after the buffing process. The CMP process involved polishing each side of the cutting surface of the blade for a total of about 2 minutes using a polishing slurry (5% alpha-alumina, mean particle size of about 350 nm, in deionized water adjusted to pH 10.5) at a flow rate of about 20 mL per minute and a non-woven polyester polishing pad (Beta Lap pad, about 0.05 inches thick; J. I. Morris, Southbridge, Mass.), at a platen speed of about 120 rpm with the blade mounted at about 45 degrees to the tangent line of the pad edge. The blade was rocked back and forth slowly (about 1 second per rock) for about 2 minutes, while pressing the blade against the moving pad to engage the full surface of the blade with the pad and CMP composition thereon. Prior to CMP, the pad was conditioned with deionized water and then CMP slurry. The CMP process was then repeated on the other side of the blade. After CMP, the blade was wiped clean with 10 percent isopropanol as described above.

The surface roughness of the blades was determined using standard surface metrology techniques, which are well known in the art, to determine the RMS surface roughness (Rq), the average surface roughness (Ra), and the peak-valley roughness (Rz) using a “cylinder and tilt” correction to account for curvature of the blade. Data was also analyzed using high-band pass filtering of the data to isolate and analyze the high-frequency noise from the overall figure (shape) of the blade. The surface roughness data for the blades are provided in Table 1, in which “HP” stands for “highly polished”.

TABLE 1

| | Cylinder and Tilt | | | High Band-Pass | | |
|---|---|---|---|---|---|---|
| Blade | Ra, nm | Rq, nm | Rz, nm | Ra, nm | Rq, nm | Rz, nm |
| Diamond, as is | 386.71 | 481.66 | 4106.96 | 30.79 | 54.14 | 1508 |
| Beaver, as is | 215.77 | 271.34 | 2525.16 | 31.32 | 51.51 | 1364.38 |
| Bard Parker, | 346.55 | 440.75 | 3459.09 | 33.23 | 60.41 | 1613.31 |
| as is | | | | | | |
| IonFusion, as is | 331.28 | 425.17 | 4269.55 | 46.72 | 77.64 | 1938.64 |
| One-Pass Buff | 83.46 | 106.60 | 1127.27 | 9.14 | 13.78 | 516.94 |
| (HP) | | | | | | |
| Multi-pass Buff | 48.05 | 63.14 | 875.29 | 5.61 | 8.97 | 390.49 |
| (HP) | | | | | | |
| Buff + CMP | 24.99 | 34.37 | 687.39 | 1.93 | 3.88 | 269.78 |
| (HP) | | | | | | |

The data in Table 1 indicate that the blades that were highly polished (HP) by either one-pass buffing, multi-pass-buffing or multi-pass buffing combined with CMP, as described above, were significantly sharper and smoother than the commercial blades as-received from the manufacturer. Such highly polished blades are suitable for use in the methods of the present invention.

### Example 2

The methods of the present invention were evaluated in a guinea pig model using female Hartley guinea pigs (about 400 grams in weight), housed and cared for according to NIH guidelines, to assess the improvement in wound healing achieved by performing surgical incisions with surgical blades having a uniform ultimate cutting edge and smooth surface (i.e., RMS surface roughness of not more than about 200 nm, and a uniform edge having a deviation along any 680 μm segment of the ultimate edge of no more than about 4 μm), according to the methods of the present invention.

The test animals were anesthetized and prepped under standard surgical conditions and two 6 cm long incisions were made on the back of each animal through the skin and the underlying muscles (panniculosis carinea). One incision was made with a standard, commercial BARD-PARKER® No. 15 blade (as-received), while the other incision was made with a highly polished BARD-PARKER® No. 15 blade of the invention having a sharp, uniform edge (as indicated by microscopic analysis and high band-pass surface roughness) as compared to the commercial blade. The incisions were closed using steel clips, spaced about one cm apart along the length of the incision, which were removed after about seven days. Groups of three animals each were sacrificed and their incisions were evaluated at 1, 2, 5, 7, 9, 16 days post surgery, as well as at 6 months post surgery. The incisions were evaluated microscopically and visually to assess the rapidity of wound closing, the inflammation, reepithelialization, granulation tissue formation, scar area, collagen deposition, and the like. Immunohistochemical staining was used to quantify macrophage infiltration and other aspects of the healing process.

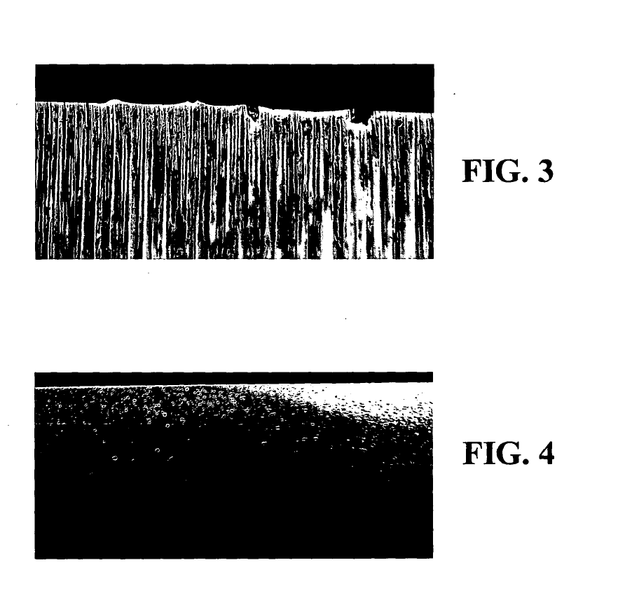

In these evaluations, the highly polished blades had an average high band-pass RMS edge roughness of about 3.9 nm, compared to 54 nm for the commercial blades (as-received). The sides of the highly polished blade were also visibly smoother than the commercial blade, i.e., see FIG. 3 (commercial blade) compared to FIG. 4 (highly polished blade). In contrast, the peak-to-valley surface roughness for the as-received commercial blades was about 3,400 nm.

Comparison of the incisions created using the standard commercial blade versus the highly polished blades indicated that wound healing was significantly improved using the methods of the invention. Among the observed benefits were the following: Wound closure time was significantly decreased (over 90 percent closure in two days, compared to 5 days for closure of the incisions made with the commercial blades). Reepithelialization increased significantly (80% in two days for the highly polished blades, versus 20% in 2 days for the commercial as-received blades). In addition, macrophage infiltration into the wounds was significantly decreased in the incisions made according to the invention compared to those made with the commercial blades (30% to 70% reduction), indicating a reduced level of inflammation. Collagen deposition was also significantly reduced in the incisions made with the highly polished blades, compared to the commercial as-received blades (about 50% reduction during days 3-14 post surgery, and up to about 90% reduction by 6 months post-surgery), indicating significantly reduced scar formation. The reduced scarring was also confirmed by histological analysis, which showed lower scar area and narrower scar width for the incisions made according to the invention compared to incisions made by as-received commercial blades.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for ameliorating tissue trauma from a surgical incision, which comprises making the surgical incision with a cutting instrument comprising a cutting instrument body defining two opposed sides and a direction of elongation, and including at least one cutting edge extending along the direction of elongation, the cutting edge defining an ultimate edge and two beveled faces adjacent the ultimate edge, wherein the cutting edge of the cutting instrument has at least one characteristic selected from the group consisting of (a) the ultimate edge has a maximum height deviation of not more than about 4 μm along any 680 μm segment thereof, (b) each beveled face adjacent the ultimate edge has a maximum height deviation of not more than about 4 μm along any 680 μm segment thereof, and (c) each beveled face adjacent the ultimate edge has a root mean square (RMS) surface roughness (Rq) of not more than about 200 nm.

2. The method of claim 1 wherein the maximum height deviation of the ultimate edge, each beveled face, or the ultimate edge and each the beveled face is not more than about 1 μm along any 680 μm segment thereof.

3. The method of claim 1 wherein the cutting edge of the cutting instrument comprises a metal, a metal oxide, a ceramic material, or a combination thereof.

4. The method of claim 1 wherein the cutting edge comprises an alloy of iron with at least one element, selected from the group consisting of carbon, chromium, nickel, and cobalt.

5. The method of claim 1 wherein the cutting edge comprises a bulk amorphous metal alloy.

6. The method of claim 1 wherein the cutting instrument is a surgical scalpel.

7. The method of claim 1 wherein the amelioration of tissue trauma includes a reduction in post-operative inflammation at the site of the surgical incision.

8. The method of claim 1 wherein the amelioration of tissue trauma includes a reduction in post-operative scar tissue at the site of the surgical incision.

9. A method for promoting healing of surgically incised tissue, which comprises making a surgical incision with a cutting instrument comprising a cutting instrument body defining two opposed sides and a direction of elongation, and including at least one cutting edge extending along the direction of elongation, the cutting edge defining an ultimate edge and two beveled faces adjacent the ultimate edge, wherein the cutting edge of the cutting instrument has at least one characteristic selected from the group consisting of (a) the ultimate edge has a maximum height deviation of not more than about 4 μm along any 680 μm segment thereof, (b) each beveled face adjacent the ultimate edge has a maximum height deviation of not more than about 4 μm along any 680 μm segment thereof, and (c) each beveled face adjacent the ultimate edge has a root mean square (RMS) surface roughness (Rq) of not more than about 200 nm.

10. The method of claim 9 wherein the maximum height deviation of the ultimate edge, each beveled face, or the ultimate edge and each the beveled face is not more than about 1 μm along any 680 μm segment thereof.

11. The method of claim 9 wherein the cutting edge of the cutting instrument comprises a metal, a metal oxide, a ceramic material, or a combination thereof.

12. The method of claim 9 wherein the cutting edge comprises an alloy of iron with at least one element selected from the group consisting of carbon, chromium, nickel, and cobalt.

13. The method of claim 9 wherein the cutting edge comprises a bulk amorphous metal alloy.

14. The method of claim 9 wherein the cutting instrument is a surgical scalpel.

15. A method for ameliorating scarring of surgically incised tissue, which comprises making a surgical incision with a cutting instrument comprising a cutting instrument body defining two opposed sides and a direction of elongation, and including at least one cutting edge extending along the direction of elongation, the cutting edge defining an ultimate edge and two beveled faces adjacent the ultimate edge, wherein the cutting edge of the cutting instrument has at least one characteristic selected from the group consisting of (a) the ultimate edge has a maximum height deviation of not more than about 4 μm along any 680 μm segment thereof, (b) each beveled face adjacent the ultimate edge has a maximum height deviation of not more than about 4 μm along any 680 μm segment thereof, and (c) each beveled face adjacent the ultimate edge has a root mean square (RMS) surface roughness (Rq) of not more than about 200 nm.

16. The method of claim 15 wherein the maximum height deviation of the ultimate edge, each beveled face, or the ultimate edge and each the beveled face is not more than about 1 μm along any 680 μm segment thereof.

17. The method of claim 15 wherein the cutting edge of the cutting instrument comprises a metal, a metal oxide, a ceramic material, or a combination thereof.

18. The method of claim 15 wherein the cutting edge comprises an alloy of iron with at least one element selected from the group consisting of carbon, chromium, nickel, and cobalt.

19. The method of claim 15 wherein the cutting edge comprises a bulk amorphous metal alloy.

20. The method of claim 15 wherein the cutting instrument is a surgical scalpel.

21. A method for ameliorating inflammation of surgically incised tissue, which comprises making a surgical incision with a cutting instrument comprising a cutting instrument body defining two opposed sides and a direction of elongation, and including at least one cutting edge extending along the direction of elongation, the cutting edge defining an ultimate edge and two beveled faces adjacent the ultimate edge, wherein the cutting edge of the cutting instrument has at least one characteristic selected from the group consisting of (a) the ultimate edge has a maximum height deviation of not more than about 4 μm along any 680 μm segment thereof, (b) each beveled face adjacent the ultimate edge has a maximum height deviation of not more than about 4 μm along any 680 μm segment thereof, and (c) each beveled face adjacent the ultimate edge has a root mean square (RMS) surface roughness (Rq) of not more than about 200 nm.

22. The method of claim 21 wherein the maximum height deviation of the ultimate edge, each beveled face, or the ultimate edge and each the beveled face is not more than about 1 μm along any 680 μm segment thereof.

23. The method of claim 21 wherein the cutting edge of the cutting instrument comprises a metal, a metal oxide, a ceramic material, or a combination thereof.

24. The method of claim 21 wherein the cutting edge comprises an alloy of iron with at least one element selected from the group consisting of carbon, chromium, nickel, and cobalt.

25. The method of claim 21 wherein the cutting edge comprises a bulk amorphous metal alloy.

26. The method of claim 21 wherein the cutting instrument is a surgical scalpel.

27. A method for promoting closure of surgically incised tissue, which comprises making a surgical incision with a cutting instrument comprising a cutting instrument body defining two opposed sides and a direction of elongation, and including at least one cutting edge extending along the direction of elongation, the cutting edge defining an ultimate edge and two beveled faces adjacent the ultimate edge, wherein the cutting edge of the cutting instrument has at least one characteristic selected from the group consisting of (a) the ultimate edge has a maximum height deviation of not more than about 4 μm along any 680 μm segment thereof, (b) each beveled face adjacent the ultimate edge has a maximum height deviation of not more than about 4 μm along any 680 μm segment thereof, and (c) each beveled face adjacent the ultimate edge has a root mean square (RMS) surface roughness (Rq) of not more than about 200 nm.

28. The method of claim 27 wherein the maximum height deviation of the ultimate edge, each beveled face, or the ultimate edge and each the beveled face is not more than about 1 μm along any 680 μm segment thereof.

29. The method of claim 27 wherein the cutting edge of the cutting instrument comprises a metal, a metal oxide, a ceramic material, or a combination thereof.

30. The method of claim 27 wherein the cutting edge comprises an alloy of iron with at least one element selected from the group consisting of carbon, chromium, nickel, and cobalt.

31. The method of claim 27 wherein the cutting edge comprises a bulk amorphous metal alloy.

32. The method of claim 27 wherein the cutting instrument is a surgical scalpel.

33. A method for promoting tissue strength in healed or healing surgically incised tissue, which comprises making a surgical incision with a cutting instrument comprising a cutting instrument body defining two opposed sides and a direction of elongation, and including at least one cutting edge extending along the direction of elongation, the cutting edge defining an ultimate edge and two beveled faces adjacent the ultimate edge, wherein the cutting edge of the cutting instrument has at least one characteristic selected from the group consisting of (a) the ultimate edge has a maximum height deviation of not more than about 4 μm along any 680 μm segment thereof, (b) each beveled face adjacent the ultimate edge has a maximum height deviation of not more than about 4 μm along any 680 μm segment thereof, and (c) each beveled face adjacent the ultimate edge has a root mean square (RMS) surface roughness (Rq) of not more than about 200 nm.

34. The method of claim 33 wherein the maximum height deviation of the ultimate edge, each beveled face, or the ultimate edge and each the beveled face is not more than about 1 μm along any 680 μm segment thereof.

35. The method of claim 33 wherein the cutting edge of the cutting instrument comprises a metal, a metal oxide, a ceramic material, or a combination thereof.

36. The method of claim 33 wherein the cutting edge comprises an alloy of iron with at least one element selected from the group consisting of carbon, chromium, nickel, and cobalt.

37. The method of claim 33 wherein the cutting edge comprises a bulk amorphous metal alloy.

38. The method of claim 33 wherein the cutting instrument is a surgical scalpel.

39. A method for promoting reepithelialization of surgically incised tissue, which comprises making a surgical incision with a cutting instrument comprising a cutting instrument body defining two opposed sides and a direction of elongation, and including at least one cutting edge extending along the direction of elongation, the cutting edge defining an ultimate edge and two beveled faces adjacent the ultimate edge, wherein the cutting edge of the cutting instrument has at least one characteristic selected from the group consisting of (a) the ultimate edge has a maximum height deviation of not more than about 4 μm along any 680 μm segment thereof, (b) each beveled face adjacent the ultimate edge has a maximum height deviation of not more than about 4 μm along any 680 μm segment thereof, and (c) each beveled face adjacent the ultimate edge has a root mean square (RMS) surface roughness (Rq) of not more than about 200 nm.

40. The method of claim 39 wherein the maximum height deviation of the ultimate edge, each beveled face, or the ultimate edge and each the beveled face is not more than about 1 μm along any 680 μm segment thereof.

41. The method of claim 39 wherein the cutting edge of the cutting instrument comprises a metal, a metal oxide, a ceramic material, or a combination thereof.

42. The method of claim 39 wherein the cutting edge comprises an alloy of iron with at least one element selected from the group consisting of carbon, chromium, nickel, and cobalt.

43. The method of claim 39 wherein the cutting edge comprises a bulk amorphous metal alloy.

44. The method of claim 39 wherein the cutting instrument is a surgical scalpel.

45. A method for ameliorating swelling during healing of surgically incised tissue, which comprises making a surgical incision with a cutting instrument comprising a cutting instrument body defining two opposed sides and a direction of elongation, and including at least one cutting edge extending along the direction of elongation, the cutting edge defining an ultimate edge and two beveled faces adjacent the ultimate edge, wherein the cutting edge of the cutting instrument has at least one characteristic selected from the group consisting of (a) the ultimate edge has a maximum height deviation of not more than about 4 μm along any 680 μm segment thereof, (b) each beveled face adjacent the ultimate edge has a maximum height deviation of not more than about 4 μm along any 680 μm segment thereof, and (c) each beveled face adjacent the ultimate edge has a root mean square (RMS) surface roughness (Rq) of not more than about 200 nm.

46. The method of claim 45 wherein the maximum height deviation of the ultimate edge, each beveled face, or the ultimate edge and each the beveled face is not more than about 1 μm along any 680 pin segment thereof.

47. The method of claim 45 wherein the cutting edge of the cutting instrument comprises a metal, a metal oxide, a ceramic material, or a combination thereof.

48. The method of claim 45 wherein the cutting edge comprises an alloy of iron with at least one element selected from the group consisting of carbon, chromium, nickel, and cobalt.

49. The method of claim 45 wherein the cutting edge comprises a bulk amorphous metal alloy.

50. The method of claim 45 wherein the cutting instrument is a surgical scalpel.

51. A method for ameliorating morbidity of surgically incised tissue, which comprises making a surgical incision with a cutting instrument comprising a cutting instrument body defining two opposed sides and a direction of elongation, and including at least one cutting edge extending along the direction of elongation, the cutting edge defining an ultimate edge and two beveled faces adjacent the ultimate edge, wherein the cutting edge of the cutting instrument has at least one characteristic selected from the group consisting of (a) the ultimate edge has a maximum height deviation of not more than about 4 μm along any 680 μm segment thereof, (b) each beveled face adjacent the ultimate edge has a maximum height deviation of not more than about 4 μm along any 680 μm segment thereof, and (c) each beveled face adjacent the ultimate edge has a root mean square (RMS) surface roughness (Rq) of not more than about 200 nm.

52. The method of claim 51 wherein the maximum height deviation of the ultimate edge, each beveled face, or the ultimate edge and each the beveled face is not more than about 1 μm along any 680 μm segment thereof.

53. The method of claim 51 wherein the cutting edge of the cutting instrument comprises a metal, a metal oxide, a ceramic material, or a combination thereof.

54. The method of claim 51 wherein the cutting edge comprises an alloy of iron with at least one element selected from the group consisting of carbon, chromium, nickel, and cobalt.

55. The method of claim 51 wherein the cutting edge comprises a bulk amorphous metal alloy.

56. The method of claim 51 wherein the cutting instrument is a surgical scalpel.

57. A highly polished cutting instrument comprising a cutting instrument body defining two opposed sides and a direction of elongation, and including at least one cutting edge extending along the direction of elongation, the cutting edge defining an ultimate edge and two beveled faces adjacent the ultimate edge, wherein each beveled face adjacent the ultimate edge has a root mean square (RMS) surface roughness (Rq) of not more than about 200 nm, and at least one of (a) the ultimate edge, and (b) each beveled face, has a maximum height deviation of not more than about 4 μm along any 680 μm segment thereof.

58. The cutting instrument of claim 57 wherein the maximum height deviation of the ultimate edge, each beveled face, or the ultimate edge and each the beveled face is not more than about 1 μm along any 680 μm segment thereof.

59. The cutting instrument of claim 57 wherein the cutting edge of the cutting instrument comprises a metal, a metal oxide, a ceramic material, or a combination thereof.

60. The cutting instrument of claim 57 wherein the cutting edge comprises an alloy of iron with at least one element selected from the group consisting of carbon, chromium, nickel, and cobalt.

61. The cutting instrument of claim 57 wherein the cutting edge comprises a bulk amorphous metal alloy.

62. The cutting instrument of claim 57 wherein the cutting instrument is a surgical scalpel.

63. A method of reducing batch-to-batch variability in the manufacture of cutting instruments having a body defining two opposed sides and a direction of elongation, and including at least one cutting edge extending along the direction of elongation, the cutting edge defining an ultimate edge and two beveled faces adjacent the ultimate edge, the method comprising polishing the cutting edge of each cutting instrument in each batch of cutting instruments in a manufacturing run to afford a cutting edge in which each beveled face adjacent the ultimate edge has a root mean square (RMS) surface roughness (Rq) of not more than about 200 nm, and at least one of (a) the ultimate edge, and (b) each beveled face, has a maximum height deviation of not more than about 4 μm along any 680 μm segment thereof.

64. The method of claim 63 wherein the maximum height deviation of the ultimate edge, each beveled face, or the ultimate edge and each the beveled face is not more than about 1 μm along any 680 μm segment thereof, for each cutting instrument in the batch.

65. The method of claim 63 wherein the cutting edge of the cutting instrument comprises a metal, a metal oxide, a ceramic material, or a combination thereof.

66. The method of claim 63 wherein the cutting edge comprises an alloy of iron with at least one element selected from the group consisting of carbon, chromium, nickel, and cobalt.

67. The method of claim 63 wherein the cutting edge comprises a bulk amorphous metal alloy.

68. The method of claim 63 wherein the cutting instrument is a surgical scalpel.

69. The method of claim 63 wherein the polishing of each cutting instrument is accomplished by the steps of:
(a) buffing the cutting edge of each cutting instrument to rapidly remove uneven material from the surfaces thereof;
(b) optionally chemically-mechanically polishing the buffed surfaces of each cutting edge to provide a desired level of surface roughness and maximum height deviation; and
(c) subsequently cleaning each cutting instrument to remove any debris left over from the buffing and polishing steps.
